(19) United States
(12) Patent Application Publication
(10) Pub. No.: US 2014/0316322 A1
(43) Pub. Date: Oct. 23, 2014

(54) WRAPPING APPARATUS

(71) Applicant: Kenneth Wilson, LeMars, IA (US)

(72) Inventor: Kenneth Wilson, LeMars, IA (US)

(21) Appl. No.: 13/868,576

(22) Filed: Apr. 23, 2013

(51) Int. Cl. A61F 15/00; A61F 15/02

(52) U.S. Cl. CPC A61F 15/002; A61F 15/02
USPC 602/42

(57) ABSTRACT

A wrapping apparatus for wrapping a sheet of a covering material from a roll onto an outer surface of a surgical site. The wrapping apparatus includes a handle and a body portion having a housing, a first end, and a second end. The housing and the first and second ends generally defining an interior region that is configured to receive the placement of the roll. The wrapping apparatus further includes first and second spindles that are configured to allow rotation of the roll relative to the handle for the dispensing of the sheet of covering material from the roll. The wrapping apparatus may also include a cap that is operably connected to the second end, and which secures the roll to the wrapping apparatus. Additionally, the wrapping apparatus may include a cutting apparatus that cuts or tears dispensed covering material away from the roll of covering material.

### BACKGROUND OF THE INVENTION

During the recovery process following surgery, at least a portion of a surgical site on a patient may, or should be, generally protected from exposure to moisture. For example, a patient may undergo a medical procedure and/or treatment at a location on a patient's body that requires the opening, removal, and/or closing of at least a portion of the patient's outer tissue or skin, also referred to as a surgical site. Moreover, such surgical procedures may involve an incision or cut into at least a portion of the outer tissue area, with the resulting open wound being treated in a manner intended to facilitate the healing of the affected tissue. For example, an open wound at the surgical site may be closed through the use of stitches, staples, and/or adhesive strips, among others.

However, the time needed for the surgical site to heal may not match other needs of the patient, such as, for example, the need to maintain at least certain aspects of hygiene, such as, for example, cleansing the patient's body. Yet, partaking in such cleansing activities before the surgical site has sufficiently healed may present a variety of health concerns, such as, for example, water, and associated materials therein or being carried by the water, from the cleansing activity entering into the surgical site through the healing wound. The ingress of such items into the surgical site may not only delay the healing of the wound, but may also increase the potential for the development of an infection in or around the not-yet-healed wound, among other potential health concerns.

To address such concerns, patients may be instructed to protect the wound from the ingress of moisture and other materials during certain activities by covering the surgical site in a covering material, such as, for example, plastic. However, to retain the plastic at a desired location relative to the surgical site, and/or to prevent the formation of gaps between the covering material and the patient's body, the covering material may need to be wrapped in a manner around a portion of the patient's body that allows for the covering material to sufficiently be engaged with, and/or compress against, the patient's body. For example, if the patient underwent knee surgery, the surgical site may include at least the exterior portion of the patient's knee area. Therefore, in an attempt to protect the surgical site and/or outer tissue from water, the covering material may be wrapped around the patient's knee area. Further, in order to prevent the wrapped covering material from slipping out of place or unwrapping, at least a portion of the covering material may secured to another portion of the covering material, the patient, and/or a garment worn by the patient, such as, for example, through the use of an adhesive material, such as, for example, tape.

### BRIEF SUMMARY OF THE INVENTION

An aspect of the invention is a wrapping apparatus for wrapping a sheet of a covering material from a roll onto an outer surface of a surgical site. The wrapping apparatus includes a handle and a body portion. The body portion includes a housing, a first end, and a second end. The housing and the first and second ends generally defining an interior region that is configured to receive the placement of the roll. The wrapping apparatus further includes a first spindle that is operably connected to the first end. At least a portion of the first spindle is configured to be received in an inner portion of a tube of the roll. The wrapping apparatus also includes a second spindle that is operably connected to the second end. At least a portion of the second spindle is configured to be received in the inner portion of the tube. The first spindle and the second spindle are configured to allow rotation of the roll relative to the handle for the dispensing of the sheet of covering material from the roll.

Another aspect of the present invention is a wrapping apparatus for wrapping a sheet of a covering material from a roll onto an outer surface of a surgical site. The wrapping apparatus includes a handle and a body portion having a housing, a first end, and a second end. The housing and the first and second ends generally define an interior region that is configured to receive the placement of the roll. The wrapping apparatus also includes a first spindle that is operably connected to the first end. At least a portion of the first spindle is configured to be received in an inner portion of a tube of the roll. Additionally, the wrapping apparatus includes a cap having a second spindle. The cap is configured to be operably secured in an aperture in the second end. At least a portion of the second spindle is configured to be received in the inner portion of the tube. The first spindle and the second spindle are configured to allow for rotation of the roll relative to the handle for the dispensing of the sheet of covering material from the roll.

A further aspect of the present invention is a wrapping apparatus for wrapping a sheet of a covering material from a roll onto an outer surface of a surgical site. The wrapping apparatus includes a handle and a body portion having a housing, a first end, a second end, and a cutting apparatus. The housing and the first and second ends generally define an interior region that is configured to receive the placement of the roll. The cutting apparatus is configured to cut or tear at least a portion of the sheet of covering material. The wrapping apparatus also includes a first spindle that is operably connected to the first end, at least a portion of the first spindle being configured to be received in an inner portion of a tube of the roll. Additionally, the wrapping apparatus includes a cap having a second spindle, the cap being configured to be operably secured in an aperture in the second end. At least a portion of the second spindle is configured to be received in the inner portion of the tube. The first spindle and the second spindle are configured to allow for rotation of the roll relative to the handle for the dispensing of the sheet of covering material from the roll.

### BRIEF DESCRIPTION OF SEVERAL VIEWS OF THE DRAWINGS

FIG. 1 illustrates a perspective view of a wrapping apparatus containing a roll of covering material according to an illustrated embodiment.

FIG. 2 illustrates a top view of the wrapping apparatus illustrated in FIG. 1.

FIG. 3 illustrates a front view of the wrapping apparatus illustrated in FIG. 1.

FIG. 4 illustrates a perspective view of a wrapping apparatus according to an illustrated embodiment.

FIG. 5 illustrates a cutting apparatus having a blade according to an illustrated embodiment.

FIG. 6 illustrates a cutting apparatus having a plurality of serrations according to an illustrated embodiment.

FIG. 7 illustrates a perspective view of a wrapping apparatus according to an illustrated embodiment.

FIG. 8 illustrates a side view of wrapping apparatus having a roll of covering material according to an illustrated embodiment.

FIG. 9 illustrates a perspective and partial exploded view of the wrapping apparatus shown in FIG. 8.

### DETAILED DESCRIPTION OF THE INVENTION

The present invention will now be described more fully with reference to the accompanying drawings, in which several embodiments are shown. This invention may, however, be embodied in many different forms and should not be construed as limited to the embodiments set forth here. Rather, these embodiments are examples of the invention, which has the full scope indicated by the language of the claims. Like numbers refer to like elements throughout.

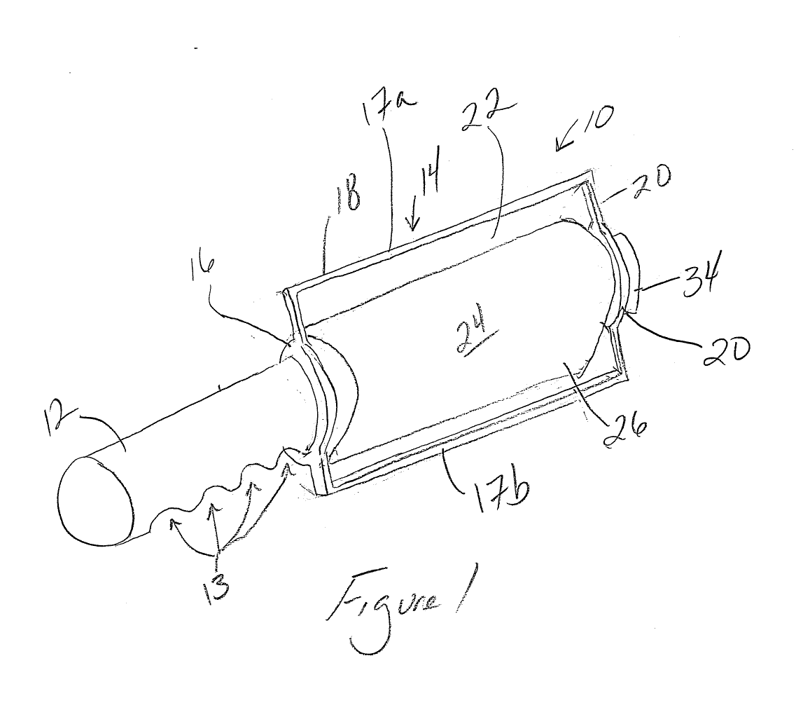

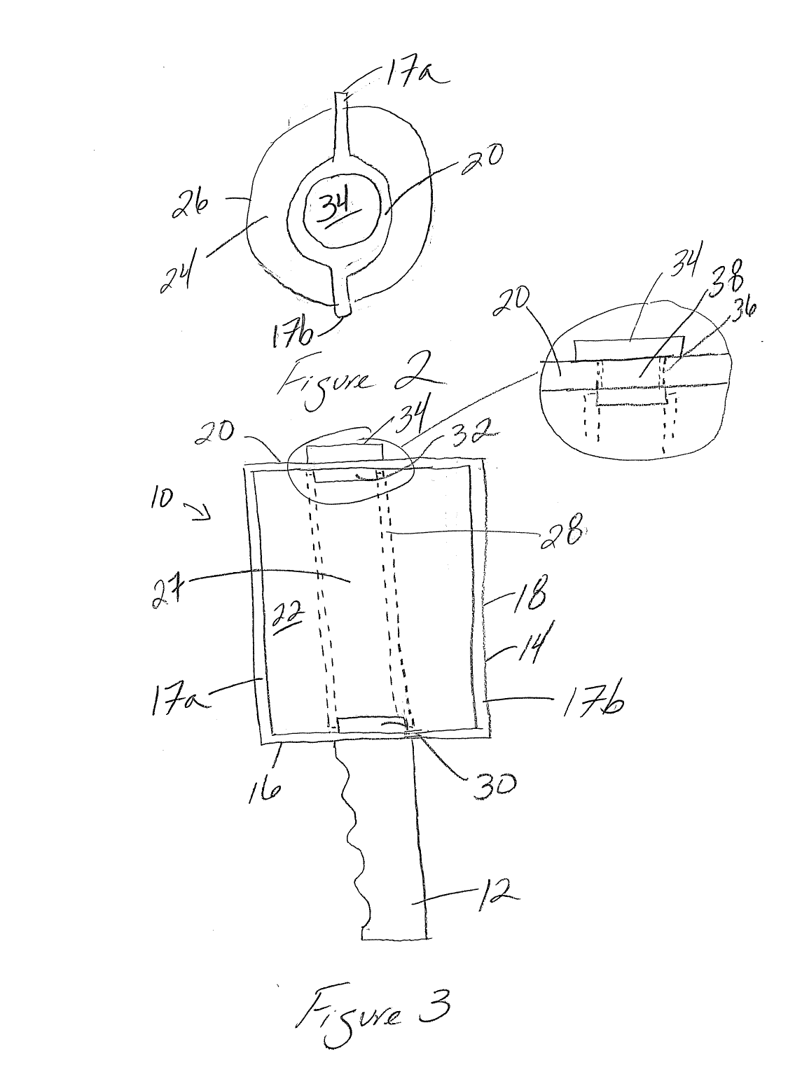

FIGS. 1-3 illustrate a wrapping apparatus 10 according to an illustrated embodiment. The wrapping apparatus 10 includes a handle 12 and a body portion 14. According to certain embodiments, the handle 12 and the body portion 14 may be a single integral unit, such as, for example, molded to form a single unit. However, according to other embodiments, the handle 12 may be operably connected to the body portion 14, such as, for example, through the use of adhesive, a plastic weld, press fit, or mechanical connectors, such as, for example, pins or screws, among other connections.

According to certain embodiments, the handle 12 may include a one or more indentations or contours 13 that may facilitate the gripping, or comfort of gripping, the handle 12 by a user. The handle 12 may terminate at a first end 16 of the body portion 14. In addition to a first end 16, the body portion 14 illustrated in FIGS. 1-3 may also include a housing 18 and a second end 20. The first end 16, housing 18, and second end 20 may generally define an interior region 22 of the body portion 14. The interior region 22 may be sized to house a roll 24 of covering material 26, such as, for example, a continuous sheet of plastic that is wrapped around a tube 28 or spindle. For example, the covering material 24 may be wrapped around a tube 28, such as, for example, a tube 28 made of cardboard. For purposes of illustration, a tube 28 for the roll 24 of covering material 26 is illustrated by dashed lines in FIG. 3.

The housing 18 may have a variety of different shapes and configurations. For example, FIG. 1 illustrates the housing 18 as have one or more arms 17a, 17b that connect the first and second ends 16, 20 while extending over a relatively small portion of the roll 24. According to certain embodiments, the arms 17a, 17b may be molded from a plastic material, such as, for example, a high impact polypropylene, and have a thickness of approximately ½ inch to ⅝ inch. However, according to other embodiments, the housing 18 may have a generally cutaway cylindrical portion that encompasses, and to an extent generally follows the shape of, at least a portion of the roll 24. Such an embodiment may shield the covering material 28 from debris during, or between, uses of the wrapping apparatus 10.

According to certain embodiments, the wrapping apparatus 10 may include first and second spindles 30, 32 that are operably connected to the first and second ends 16, 20, respectively. The first and second spindles 30, 32 may be configured to mate with the tube 28 in a manner that allows the tube 28 to be rotated relative to the handle 12 so that covering material 26 may be dispensed from the roll 24 during use of the wrapping apparatus 10. According to certain embodiments, the first spindle 30 may be an inward projection that extends from the first end 16 and into the interior region 22. Further, during use, at least portion of the first spindle 30 is configured to extend into an interior portion 27 of the tube 28. The first and second spindles may or may not be configured to rotate with the roll 24 as a sheet of the covering material 26 is dispensed from the roll 24.

According to certain embodiments, the second spindle 32 may extend from a cap 34 that is used to secure the roll 24 to the wrapping apparatus 10. More specifically, according to certain embodiments, when a roll 24 is to be secured in the interior region 22 of the wrapping apparatus 10, the cap 34 may be removed from the wrapping apparatus 10, thereby preventing the second spindle 32 from interfering with the placement of the roll 24 into the interior region 22 and into operable engagement with the first spindle 30. When the roll 24 is positioned in the interior region 22 such that at least a portion of the first spindle 32 extends into the tube 28, a portion of the cap 34 may be received in an aperture 36 in the second end 20.

According to certain embodiments, the cap 34, which may be constructed from plastic, may include a hub portion 38 that is configured to engage the aperture 36 that retains the cap 34 on the second end 20. For example, the hub portion 38 may have an external thread that mates an internal thread of the aperture 36. Alternatively, the hub portion 38 may be sized for a press fit in the aperture 36 to retain the cap 34 on the second end 20. The hub portion 38 may have the same or different size and shape than at least a portion of the second spindle 32. For example, according to certain embodiments, the second spindle 32 may be an extension or continuation of the hub portion 38. When the roll 24 is to be secured to the wrapping apparatus 10, the cap 34 and roll 24 may be positioned for at least a portion of the second spindle 32 to be received by the tube 28. Before dispensing covering material 26 from the roll 24, the cap 34 may be secured to the second end 20 so as to secure the roll 24 to the wrapping apparatus 10.

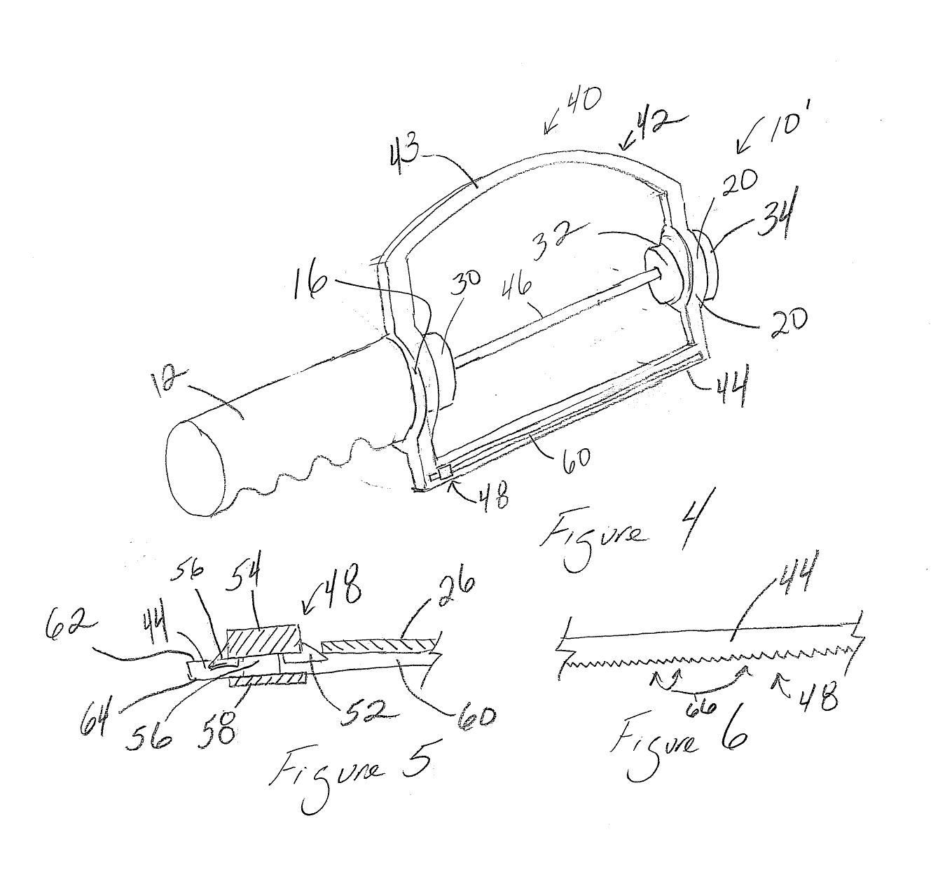

FIG. 4 illustrates a perspective view of a wrapping apparatus 10′ in which the housing 40 of the body portion 42 includes a first arm 43 and a second arm 44 that extend between the first end 16 and the second end 20. The first arm 43 may be configured to provide stability and/or strength to the wrapping apparatus 10′. Further, the first and second arms 43, 44 may have a variety of different configurations, including, for example, having a straight or curved profile, as shown in FIG. 4. Further according to certain embodiments, the shape and/or configuration of the first arm 43 and second arm 44 may be generally the same or different.

Similar to the embodiment illustrated in FIGS. 1-3, the wrapping apparatus 10′ may include a first spindle 30 and a second spindle 32 that are configured to engage the tube 28 of the roll 24. Optionally, according to certain embodiments, a guide tube 46 may extend between the first and second spindles 30, 32. The guide tube 46 may be configured to provide stability to the wrapping apparatus 10, 10′.

The wrapping apparatus 10′ may also include a cutting apparatus 48 that is configured to tear, cut, or otherwise separate covering material 48 that has been dispensed from the roll 24 from covering material 48 that remains wrapped or otherwise bound to the roll 24. Referencing FIGS. 4 and 5, according to certain embodiments, the cutting apparatus 48 may include a cutter 50 having one or more blades 52. Further, according to certain embodiments, the cutter 50 may include at least one upper leg 54, a body 56 and at least one lower leg 58. The cutter 50 may be configured to be slid or otherwise displaced along a track 60 that is positioned in the housing 40. For example, according to certain embodiments, the track 60 may be positioned along at least a portion of the second arm 44. The upper and lower legs 54, 58 may be positioned on, and extend along, opposing surfaces of the second arm 44 so as to retain the body 56 in the track 60. For example, the upper leg 54 may be positioned along an upper surface of the second arm 44 and the lower leg 56 positioned along a bottom surface 64. When a portion of the sheet of covering material 26 is to be cut, the covering material 26 may be extended over the track at a location where the cut is to occur. The cutting apparatus 48 may then be slid or displaced along the track 60, with at least one blade 52 cutting the covering material 26 that is in the path of the blade 52. The use of two or more blades 50, or a single blade 50 having at least two opposing cutting surfaces, may provide the ability for the cutter 50 to cut covering material 26 in either direction as the cutter is displaced along the track 60.

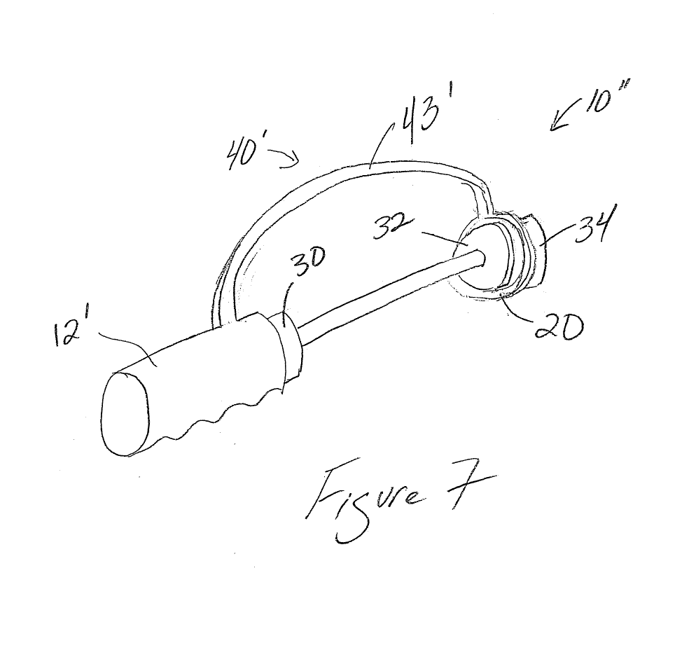

Alternatively, referencing FIG. 6, the cutting apparatus 48 may be a plurality of serrations 66 or cutters that extend from the surface of the housing 40. For example, according to certain embodiments, an edge of the housing 40, such as an edge of the second arm 44, may be molded or formed with serrations 66 that may be used to tear or cut the covering material 26. Alternatively, the serrations may be operably attached to the housing 40, such as, for example, by an adhesive, pin, or other mechanical fastener.

FIG. 7 illustrates a wrapping apparatus 10″ according to an illustrated embodiment in which one or more arms 43′ extends from the handle 12′. The arm(s) 43′ may have a variety of similar or dissimilar different configurations, such as, for example, being curved (as shown in FIG. 7) or non-curved. Such a configuration may improve the stability of the apparatus 10″. Additionally, such a configuration may allow for the handle 12′ to include and/or terminate at, the first end 16 of the body portion 14. Further, according to certain embodiments, the one or more arms 43′ may also include a cutting apparatus 48.

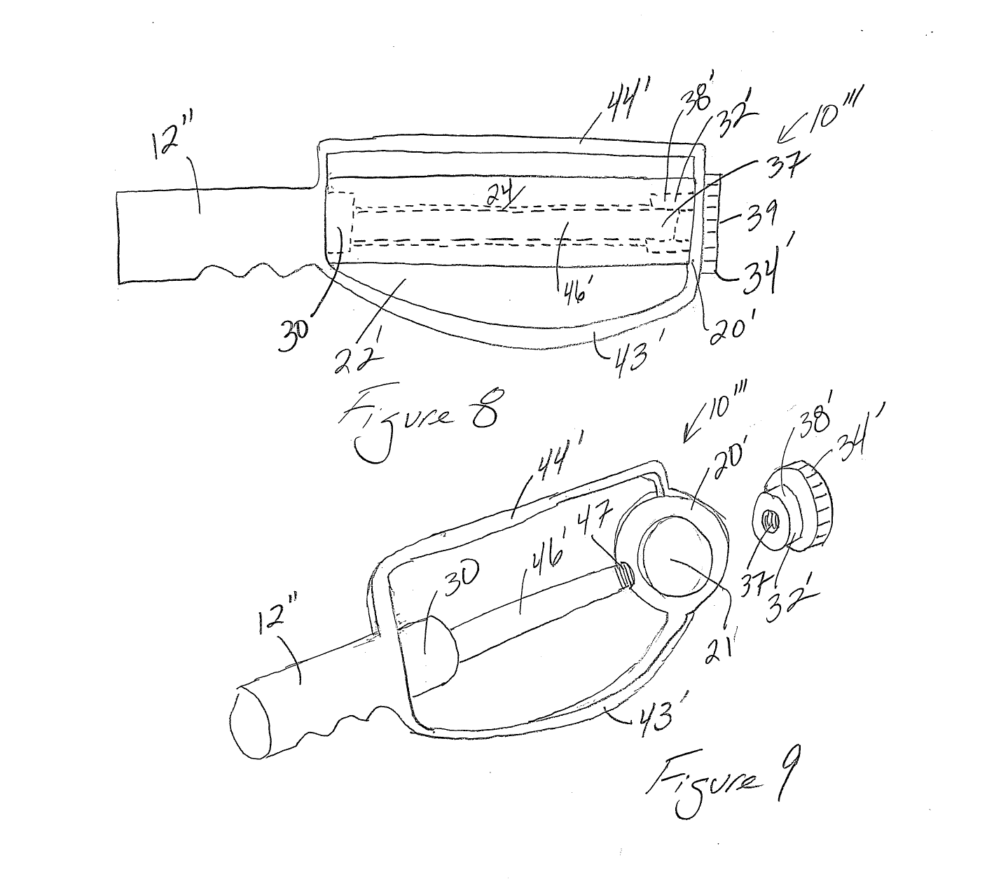

FIGS. 8 and 9 illustrate another wrapping apparatus 10′″ according to an illustrated embodiment in which two arms 43′, 44′ extend from the handle 12″. As shown, a first spindle 30 extends from the handle 12″ and into the interior region 22′. According to certain embodiments, a second spindle 32′ may be provided by a hub portion 38′ of the cap 34′. According to such an embodiment, the second end 20′ may include an orifice 21 that is sized to allow for the removable insertion of the roll 24 containing covering material into the interior region 22 of the wrapping apparatus 10′″. Additionally, the orifice 21 may also be sized to allow at least a portion of the hub portion 38′ to be positioned in the interior portion 22′, particularly if a portion of the hub portion 38′ is providing the second spindle 32′.

According to certain embodiments, the hub portion 38′ may include an external thread that mates with an internal thread that is positioned about the orifice 21 on the second end 20′. Further, according to certain embodiments, the cap 34′ may include a threaded aperture 37 that mates an external thread 47 on the guide tube 46′. Alternatively, at least a portion of the hub portion 38′ may have an external thread that mates an internal thread of an aperture in the guide tube 46′. Additionally, although the top portion 39 of the cap 34′ is illustrated as extending beyond the second end 20′, according to certain embodiments, the second end 20′ and/or the cap 34′ may be configured for the cap 38′ to be recessed in the second end 20′ when attached to the wrapping apparatus 10″.

1. A wrapping apparatus for wrapping a sheet of a covering material from a roll onto an outer surface of a surgical site, the wrapping apparatus comprising:
a handle;
a body portion having a housing, a first end, and a second end, the housing and the first and second ends generally defining an interior region, the interior region configured to receive the placement of the roll;
a first spindle operably connected to the first end, at least a portion of the first spindle configured to be received in an inner portion of a tube of the roll; and
a second spindle operably connected to the second end, at least a portion of the second spindle configured to be received in the inner portion of the tube, the first spindle and the second spindle being configured to allow rotation of the roll relative to the handle for the dispensing of the sheet of covering material from the roll.

2. The wrapping apparatus of claim 1, further including a cap configured to be removably secured to the second end, and wherein the cap includes in the second spindle.

3. The wrapping apparatus of claim 2, wherein the cap is removably secured to the second end by a threaded engagement between the cap and an orifice in the second end.

4. The wrapping apparatus of claim 2, wherein the cap is removably secured to the second end by a press fit engagement between a hub portion of the cap and an orifice in the second end.

5. The wrapping apparatus of claim 2, wherein the housing includes a first arm and a second arm.

6. The wrapping apparatus of claim 5 wherein the first arm has a generally curved shape that is different than the shape of the second arm.

7. The wrapping apparatus of 5, wherein the first arm and/or second arm includes a cutting apparatus, the cutting apparatus configured to tear or cut a dispensed portion of the sheet of covering material.

8. The wrapping apparatus of claim 7, wherein the cutting apparatus includes at least one blade, and wherein the cutting apparatus is configured to be slid along a track.

9. The wrapping apparatus of claim 7, wherein the cutting apparatus includes a plurality of serrations along at least a portion along the first arm and/or second arm.

10. A wrapping apparatus for wrapping a sheet of a covering material from a roll onto an outer surface of a surgical site, the wrapping apparatus comprising:
a handle;
a body portion having a housing, a first end, and a second end, the housing and the first and second ends generally defining an interior region, the interior region configured to receive the placement of the roll;
a first spindle operably connected to the first end, at least a portion of the first spindle configured to be received in an inner portion of a tube of the roll; and
a cap having a second spindle, the cap configured to be operably secured in an aperture in the second end, at least a portion of the second spindle configured to be received in the inner portion of the tube, the first spindle and the second spindle being configured to allow rotation of the roll relative to the handle for the dispensing of the sheet of covering material from the roll.

11. The wrapping apparatus of claim 10, wherein the cap is removably secured to the second end by a threaded engagement between the cap and an orifice in the second end.

12. The wrapping apparatus of claim 10, wherein the cap is removably secured to the second end by a press fit engagement between a hub portion of the cap and an orifice in the second end.

13. The wrapping apparatus of claim 10, wherein the housing includes a first arm and a second arm.

14. The wrapping apparatus of claim 13 wherein the first arm has a generally curved shape that is different than the shape of the second arm.

15. The wrapping apparatus of 13, wherein the first arm and/or second arm includes a cutting apparatus, the cutting apparatus configured to tear or cut a dispensed portion of the sheet of covering material.

16. The wrapping apparatus of claim 15, wherein the cutting apparatus includes at least one blade, and wherein the cutting apparatus is configured to be slid along a track.

17. The wrapping apparatus of claim 15, wherein the cutting apparatus includes a plurality of serrations along at least a portion along the first arm and/or second arm.

18. A wrapping apparatus for wrapping a sheet of a covering material from a roll onto an outer surface of a surgical site, the wrapping apparatus comprising:
a handle;
a body portion having a housing, a first end, a second end, and a cutting apparatus, the housing and the first and second ends generally defining an interior region, the interior region configured to receive the placement of the roll, the cutting apparatus configured to cut or tear at least a portion of the sheet of covering material;
a first spindle operably connected to the first end, at least a portion of the first spindle configured to be received in an inner portion of a tube of the roll; and
a cap having a second spindle, the cap configured to be operably secured in an aperture in the second end, at least a portion of the second spindle configured to be received in the inner portion of the tube, the first spindle and the second spindle being configured to allow for rotation of the roll relative to the handle for the dispensing of the sheet of covering material from the roll.

19. The wrapping apparatus of claim 18, wherein the cutting apparatus includes at least one blade, and wherein the cutting apparatus is configured to be slid along a track.

20. The wrapping apparatus of claim 18, wherein the cutting apparatus includes a plurality of serrations along at least a portion along the first arm and/or second arm.
